(19) United States
(12) Patent Application Publication
(10) Pub. No.: US 2021/0316062 A1
(43) Pub. Date: Oct. 14, 2021

(54) MODULAR RADIO-LABELING TRACER SYNTHESIZER

(71) Applicant: Dana-Farber Cancer Institute, Inc., Boston, MA (US)

(72) Inventors: Erol BARS, Exeter, NH (US); Stephen DRAGOTAKES, Medfield, MA (US); Anthony BELANGER, Newton, MA (US)

(21) Appl. No.: 17/272,106

(22) PCT Filed: Aug. 27, 2019
(86) PCT No.: PCT/US2019/048328

§ 371 Date: Feb. 26, 2021

Related U.S. Application Data
(60) Provisional application No. 62/723,226, filed on Aug. 27, 2018.

(51) Int. Cl. A61M 5/00; G21H 5/02; A61M 5/178

(52) U.S. Cl. CPC A61M 5/007; G21H 5/02; A61M 2205/051; A61M 2205/50; A61M 5/1785

(57) ABSTRACT

A modular radio-labeling tracer synthesizer system comprising a housing having a plurality of slots containing syringe actuators. Each syringe actuator including a syringe holder, a syringe driver for driving a syringe plunger in a loading and/or dispensing direction. The unit is capable of adopting multiple configurations and is fully programmable and provides enhanced flexibility in development of novel radiotracers.

### CROSS-REFERENCE TO RELATED SUBJECT MATTER

This application claims the benefit of U.S. Provisional Application No. 62/723,226, filed Aug. 27, 2018, which is hereby incorporated by reference in its entirety.

### BACKGROUND OF THE DISCLOSED SUBJECT MATTER

### Field of the Disclosed Subject Matter

The subject matter disclosed herein relates generally to radioisotopes used in medical imaging, and more particularly to systems, methods, and an apparatus for preparing the radioisotope to be used in, e.g., a medical imaging procedure. Particularly, the present disclosed subject matter includes a radio-labeling tracer synthesizer capable of multiple configurations and fully programmable for development of novel compounds and synthesis methods for use in a variety of fields, e.g., molecular imaging.

### Description of Related Art

When employed in imaging procedures, an individual dose of a premeasured radioisotope or radioisotope is administered to a subject. The individual premeasured radioisotope is prepared by a radioisotope supplier using a cyclotron to prepare the radioisotope. The radioisotope is delivered to a medical facility that administers the individual premeasured radioisotope as a radiopharmaceutical.

The process of radioisotope production in a cyclotron includes irradiating a target material, such as water, in the cyclotron with a beam of protons or deuterons to produce a desired amount of radioactivity in the target material. Typically, the cyclotron is located in a dedicated room. Examples of cyclotron produced radioisotopes include nitrogen-13, fluorine-18, carbon-11 and oxygen-15.

Often, compounds are bond to the radioactive water to produce radioisotopes such as fluorodeoxyglucose (FDG) which is produced using fluorine-18. Other radioisotopes include nitrogen-13 ammonia which is used in myocardial applications, carbon-11 tracers which are commonly used in neurologic applications; and oxygen-15 gas as well as tracers derived from it which are commonly used in blood flow applications. More specifically, the radioactive water is typically delivered to a separate room that includes a synthesizing device for bonding the compound to the radioactive water and a dispensing station for dividing the radioisotope into individual doses that are stored in individual vials or containers.

The present disclosure provides a novel system, and corresponding method, of synthesizing radioisotopes.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a radio labeling tracer synthesizer capable of multiple configurations and fully programmable, which provides development of novel compounds and synthesis methods for use in a variety of fields, including molecular imaging. The novel modular design permits new cancer radiotracers to be created in an efficient and safe manner. The software and hardware embodied in the present disclosure follow current Good Manufacturing Practice (cGMP) rules and regulations of the Food and Drug Administration (FDA).

The disclosed subject matter also includes a modular radio-labeling tracer synthesizer system comprising: a housing, the housing having at least one slot; at least one syringe actuator, the at least one syringe actuator disposed within the slot and removably attached to the housing; and at least one servo motor and at least one rotary valve, the at least one servo motor and at least one rotary valve removably attached to the housing.

In some embodiments, the at least one syringe actuator is attached via magnet(s) and the at least one rotary valve is removably attached to the at least one servo motor.

In some embodiments, the at least one syringe actuator includes a syringe driver configured to engage a syringe plunger for displacement in a loading and dispensing direction.

In some embodiments, the at least one syringe actuator includes a syringe holder, the syringe holder configured to receive a variety of syringe sizes.

In some embodiments, the syringe holder includes a door which can move from an open position to a closed position.

In some embodiments, the housing includes a stopper, the stopper limiting displacement of the syringe driver.

In some embodiments, the housing includes fourteen slots, with a syringe actuator disposed in each slot.

In some embodiments, the housing includes nine rotary valves, each rotary valve(s) has seven positions.

In some embodiments, the housing further comprises dual temperature controlled reactor vessels, a cooling element(s), a compressor, at least one radiation detector, and at least one programmable microprocessor.

In accordance with another aspect of the disclosure, a syringe actuator is provided comprising: a syringe holder, a syringe driver, and a manifold. The manifold having: a pump source, a vacuum source, a first conduit in fluid communication with the pump source, a second conduit in fluid communication with a vacuum source, and a switch valve, the switch valve configured to direct fluid flow through at least one of the conduits.

In some embodiments, a third conduit connects the switch valve and pump source in fluid communication.

In some embodiments, a forth conduit connects the switch valve and vacuum source in fluid communication.

In some embodiments, the syringe actuator includes a syringe driver configured to engage a syringe plunger for displacement in a loading and dispensing direction.

In some embodiments, the syringe actuator includes a visual indicator depicting the direction of displacement of the syringe plunger.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1-2 are schematic representations of an exemplary cyclotron systems which can be employed in connection with the radioisotope production system disclosed herein.

FIGS. 3-14 are schematic representations of differing orientations including isometric, side, top, bottom, front and rear views of an exemplary modular synthesizer in accordance with the disclosed subject matter.

FIGS. 15-19 are schematic representations of the housing of the exemplary modular synthesizer (with remaining components omitted for clarity) in accordance with the disclosed subject matter.

FIGS. 20-21 are photographs of the exemplary modular synthesizer in accordance with the disclosed subject matter.

FIGS. 22-23 are schematic representations of an exemplary dual reactor of the modular synthesizer in accordance with the disclosed subject matter.

FIGS. 24-33 are schematic representations of differing orientations including isometric, side, top, bottom, front and rear of an exemplary syringe actuator of the modular synthesizer in accordance with the disclosed subject matter.

FIG. 34 is a schematic representations of an exemplary motor and valve configuration in accordance with the disclosed subject matter.

FIGS. 35-36B are schematic representations of an exemplary graphical user interface of the modular synthesizer in accordance with the disclosed subject matter.

FIGS. 37-40 are schematic representations of the exemplary syringe actuators of the modular synthesizer in accordance with the disclosed subject matter.

### DETAILED DESCRIPTION OF AN EXEMPLARY EMBODIMENT

Reference will now be made in detail to exemplary embodiments of the disclosed subject matter, an example of which is illustrated in the accompanying drawings. The method and corresponding steps of the disclosed subject matter will be described in conjunction with the detailed description of the system.

The present disclosure is directed towards a radioisotope production system that receives the output from a cyclotron, which is a type of particle accelerator in which a beam of charged particles (e.g., H-charged particles or D-charged particles) are accelerated outwardly along a spiral orbit. The cyclotron directs the beam into a target material to generate the radioisotopes (or radionuclides). Cyclotrons are known in the art, and an exemplary cyclotron is disclosed in U.S. Pat. No. 10,123,406, the entirety, including structural components and operational controls, is hereby incorporated by reference.

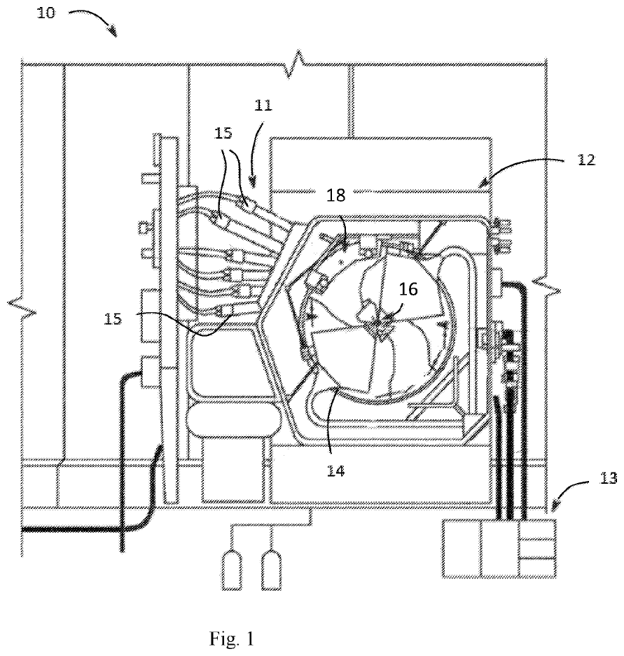

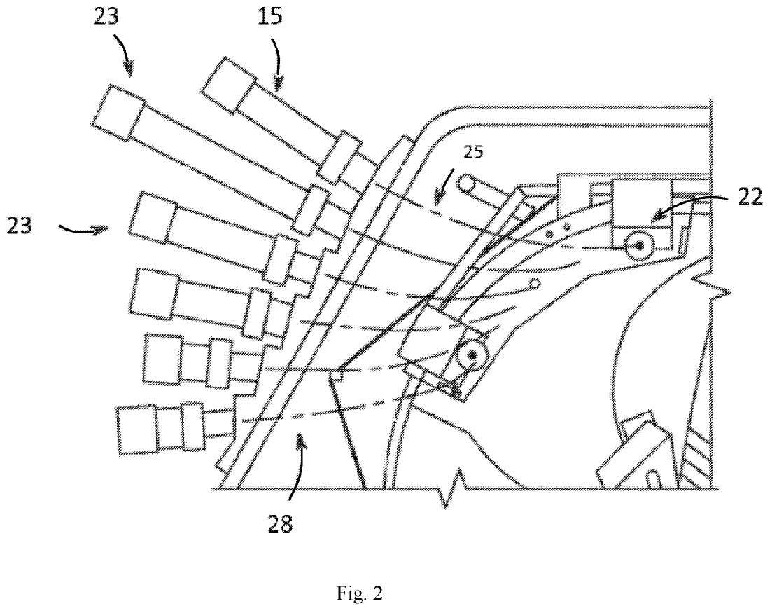

For example, FIG. 1 depicts an exemplary cyclotron construction in which the particle beam is directed by the radioisotope production system 10 through the extraction system 18 along a beam transport path and into the target system 11 so that the particle beam is incident upon the designated target material (solid, liquid or gas). In this exemplary configuration, the target system 11 includes six potential target locations 15, however a greater/lesser number of target locations 15 can be employed. Similarly, the relative angle of each target location 15 relative to the cyclotron body can be varied (e.g. each target location 15 can be angled over a range of 0°˜90° with respect to a horizontal axis in FIG. 2). Additionally, the radioisotope production system 10 and the extraction system 18 can be configured to direct the particle beam along different paths toward the target locations 15.

FIG. 2 is a zoom-in side view of the extraction system 18 and the target system 11. In the illustrated embodiment, the extraction system 18 includes first and second extraction units 22. The extraction process can include stripping the electrons of the charged particles (e.g., the accelerated negative charged particles) as the charged particles pass through an extraction foil—where the charge of the particles is changed from a negative charge to a positive charge thereby changing the trajectory of the particles in the magnet field. Extraction foils may be positioned to control a trajectory of an external particle beam 25 that includes the positively-charged particles and may be used to steer the external particle beam 25 toward designated target locations 15.

The present disclosure provides rapid synthesis times and is fully configurable to suit the development of any new radioactive compound. The system uses commercially available consumables, thus reducing setup cost. Additionally, the present synthesis system can be employed with a wide range of radio metal isotopes configured as sold, liquid or gas targets.

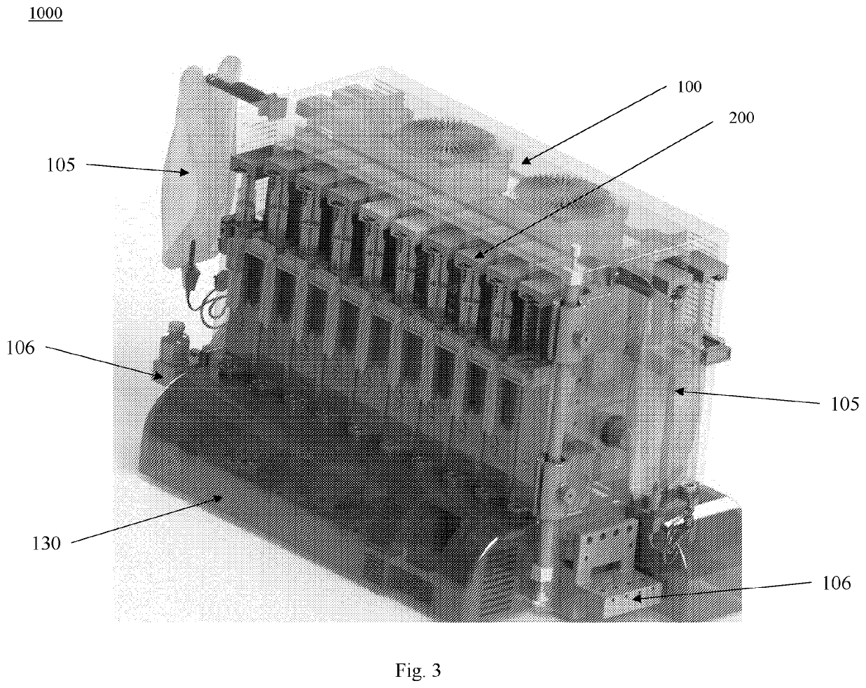

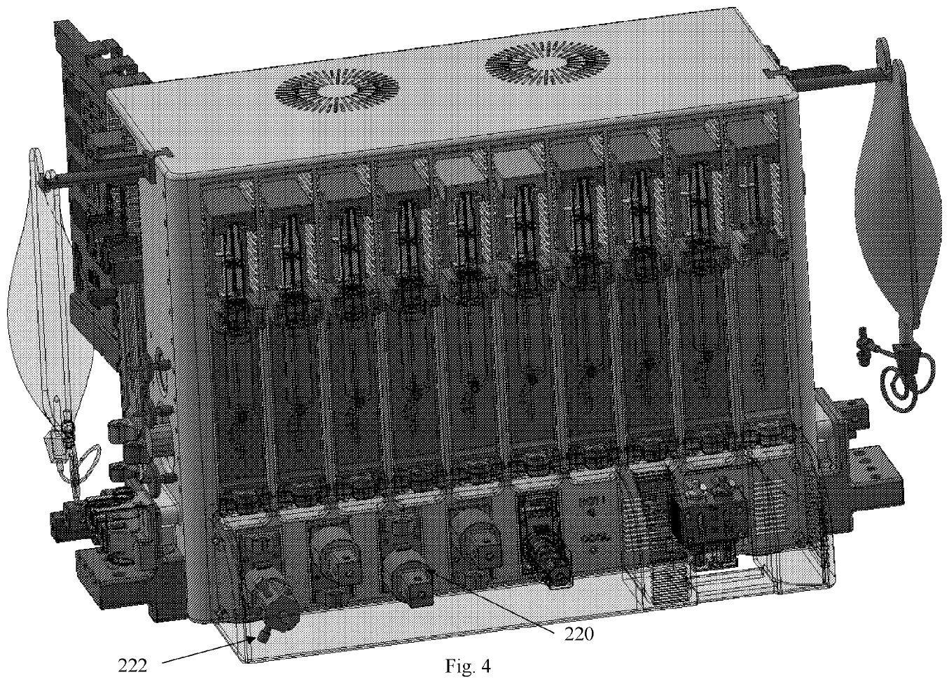

As shown in FIG. 3, the system 1000 generally includes a modular radio-labeling tracer synthesizer including a housing 100, syringe actuator(s) 200, corresponding valves, motors, tubing, etc., which is capable of multiple configurations and can be assembled by hand, without use of any tooling. Each component is described in further detail below.

Synthesizer Housing

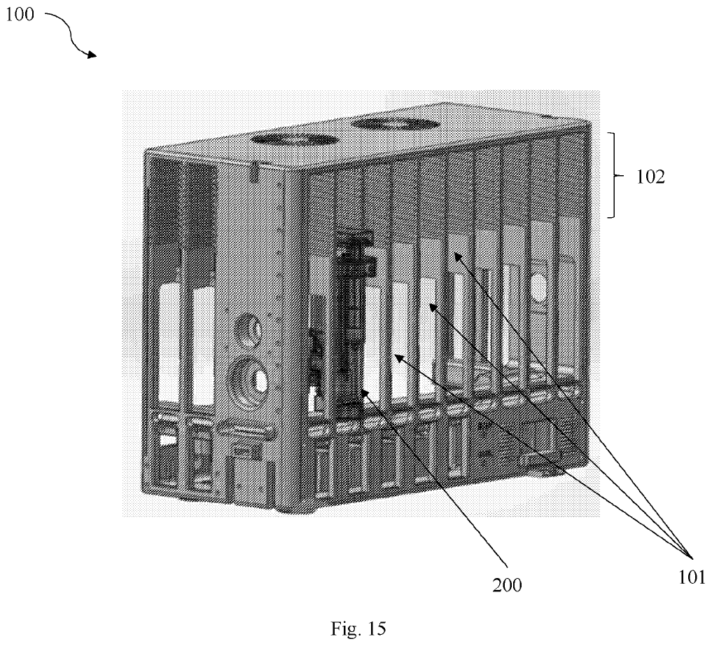

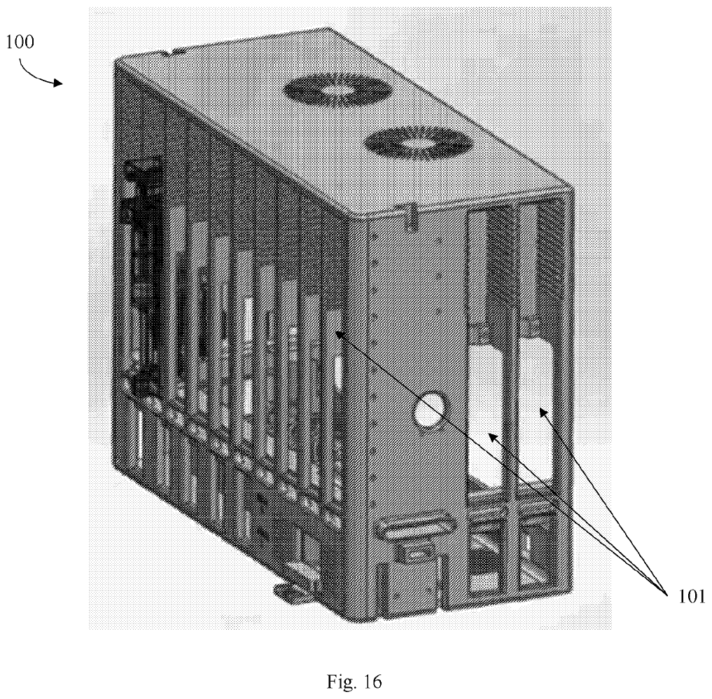

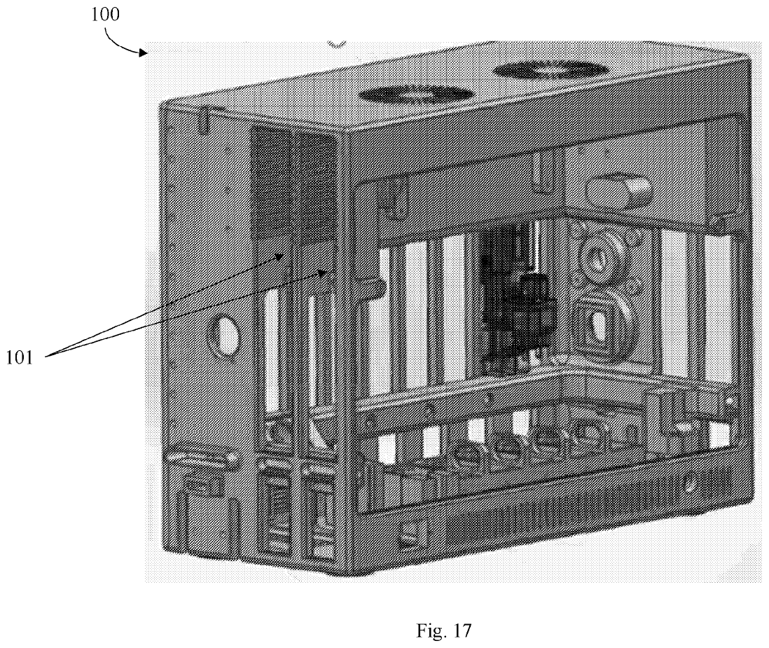

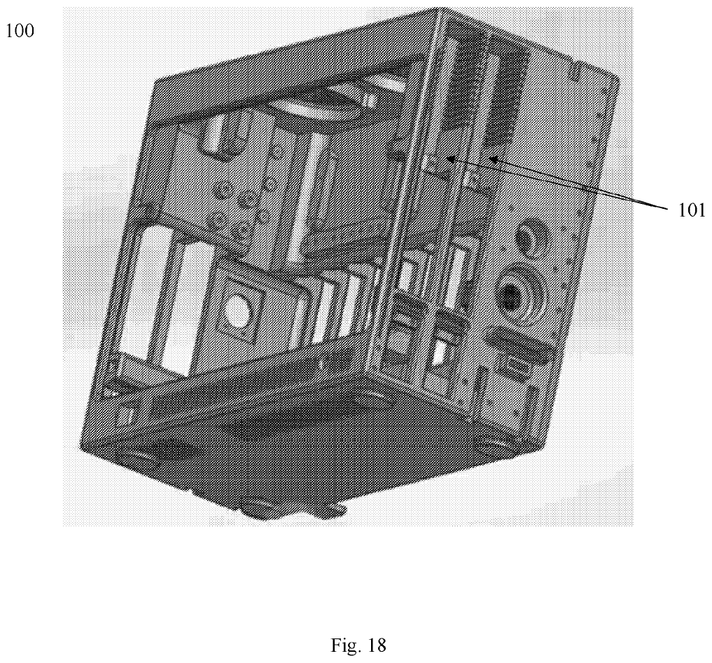

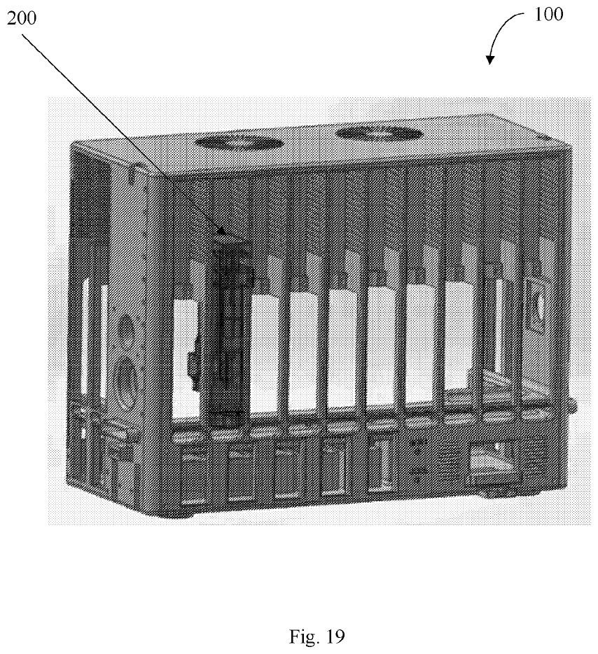

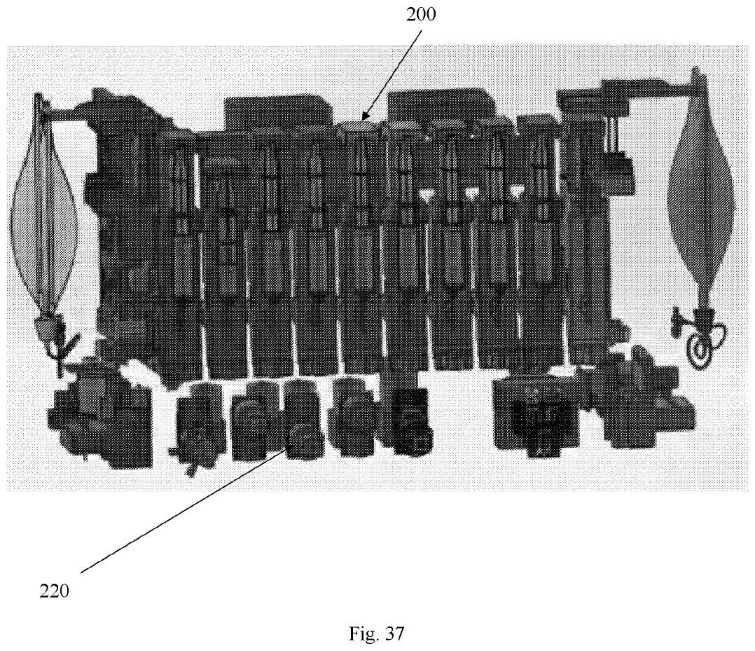

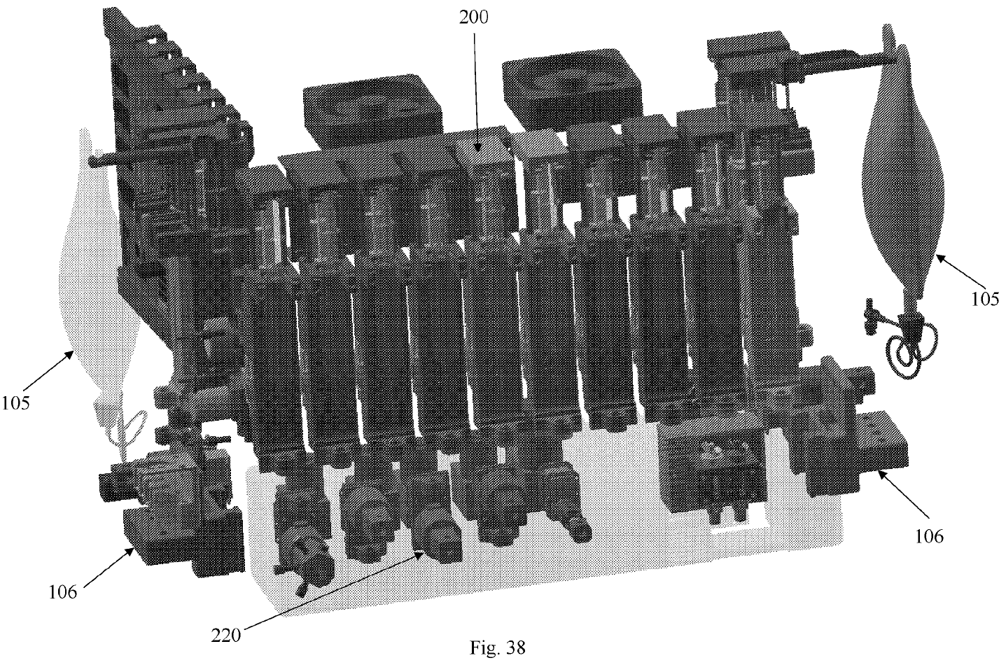

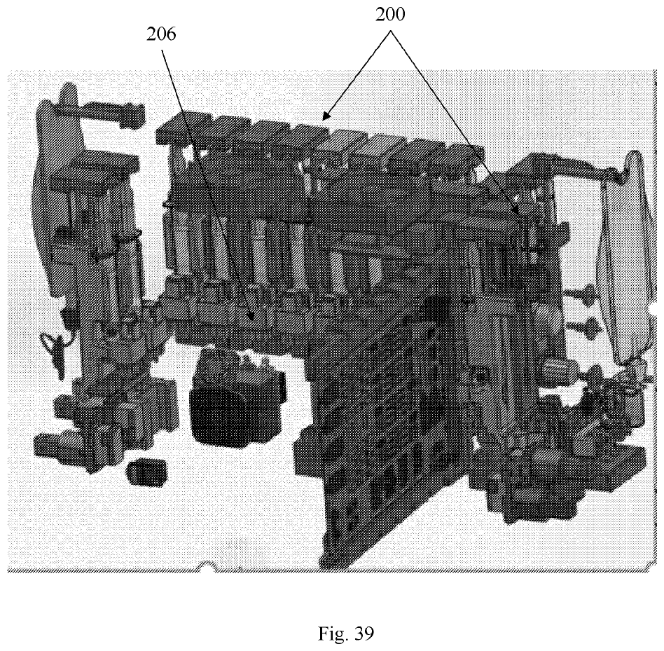

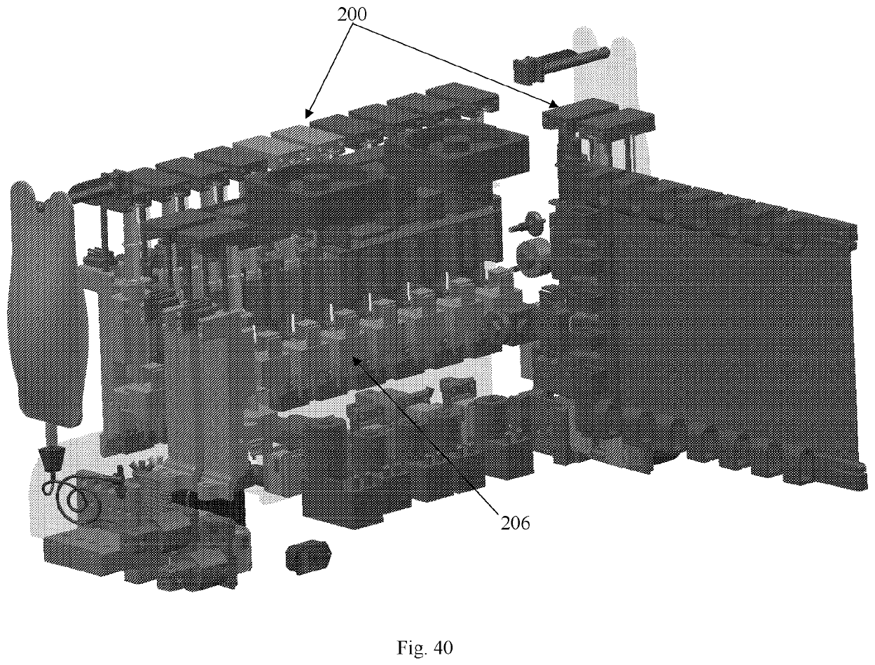

In accordance with an aspect of the present disclosure, a housing 100 is provided which allows for a modular synthesizer design capable of multiple configurations. In the exemplary embodiment depicted, the housing 100 includes slots or channels for incorporating fourteen linear syringe actuators and nine rotary valves, though artisans of ordinary skill will understand that additional/alternative configurations are within the scope of the disclosure, and the housing 100 can be scaled up/down as desired to accommodate the particular configuration required. For sake of clarity, FIGS. 15-19 depict the housing with all other components removed (in contrast, FIGS. 37-40 depict all the components of the system, without the housing). The housing can be manufactured by additive manufacturing (i.e. 3-D printing) using a laser as the power source to sinter powdered material (e.g. nylon, polyamide, etc.), aiming the laser automatically at points in space defined by a 3D housing model, and binding the material together to create a solid structure. Additionally or alternatively, certain sections/components of the synthesizer system can be formed of aluminum and plastic using Direct Metal Laser Sintering (DMLS) and Fused Deposition Modeling (FDM).

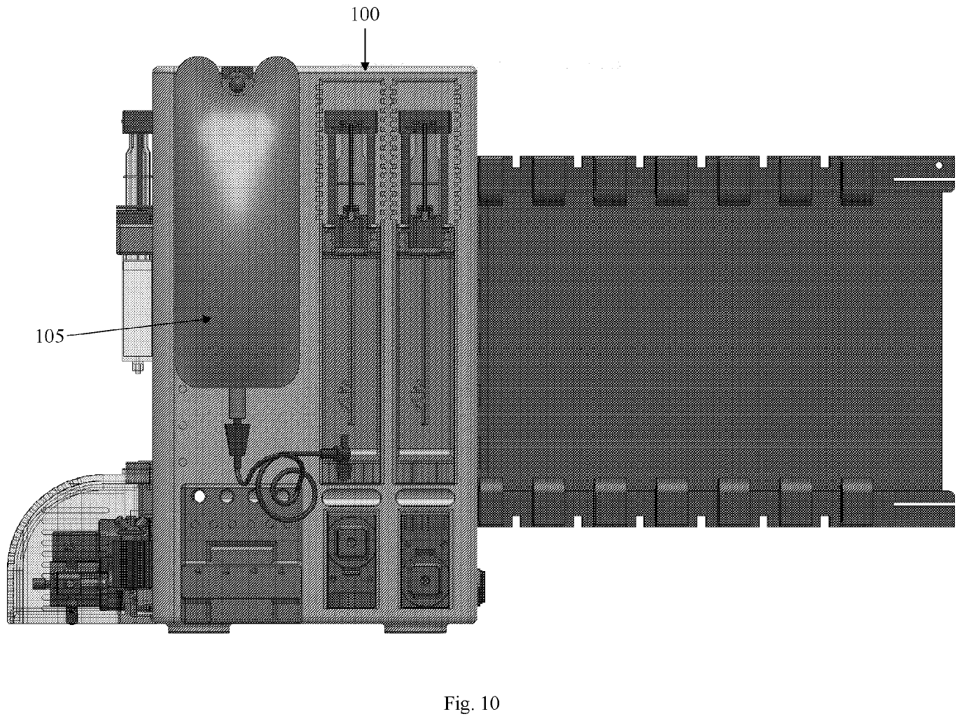

As shown in FIGS. 15-19, the slots 101 extend vertically within the housing and are sized to receive the modular syringe actuators 200 (described in further detail below). In this exemplary embodiment, ten slots 101 are provided on the front face of the housing, with two slots provided on the left face, and two slots provided on the right face (thus equaling fourteen total slots). The upper region of the slots 101 include a plurality of notches or slats 102 which can receive a shelf-like stopper 103 (see FIG. 10) which prevents the syringe plunger from extending beyond a predetermined distance (e.g. prevents the plunger from being pulled out of the syringe barrel when the synthesizer system is operating to extend the plunger and draw/load contents into the syringe barrel). In the exemplary embodiment shown, the slots 101 have uniform dimensions, however slots of varying width, height and depth can be included, if desired.

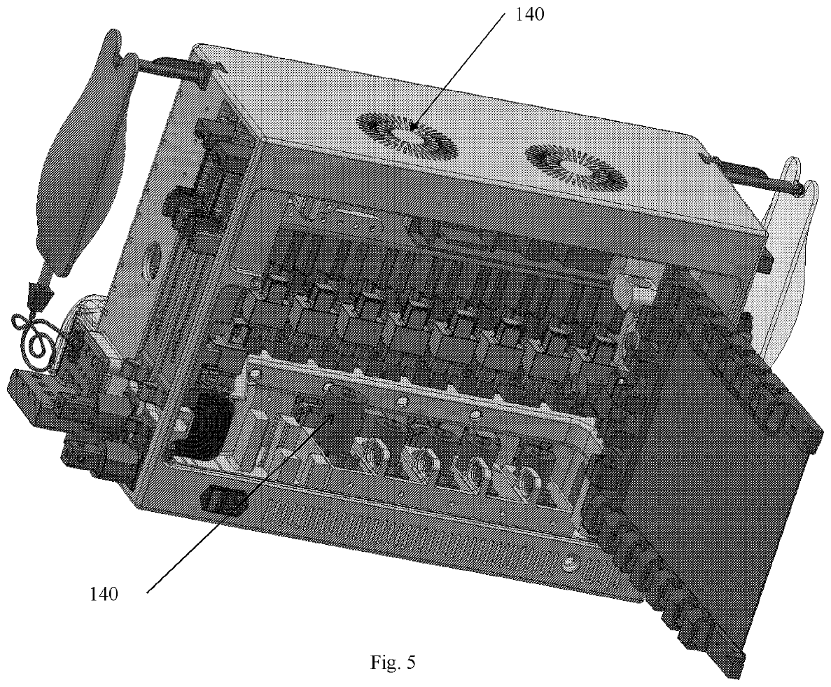

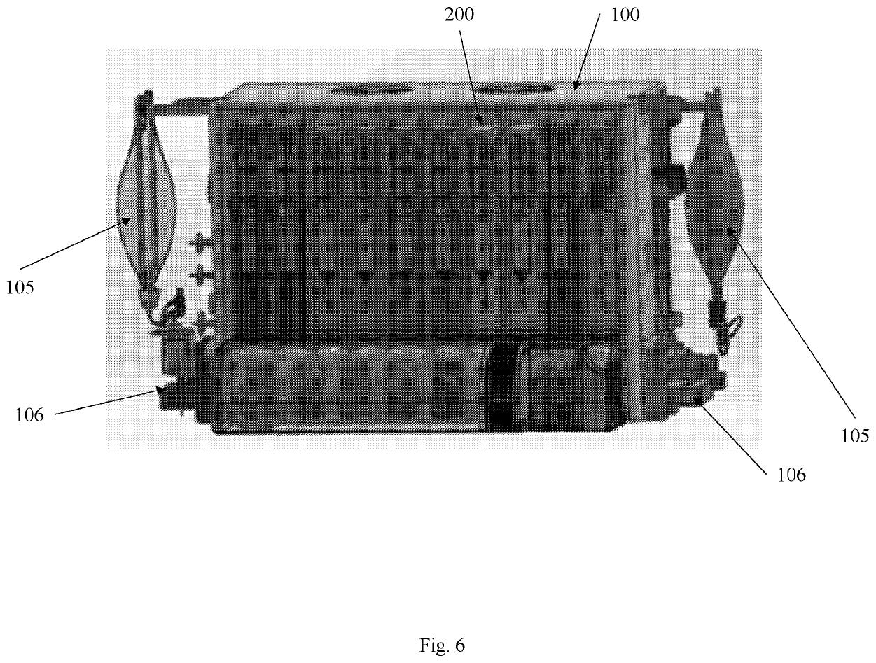

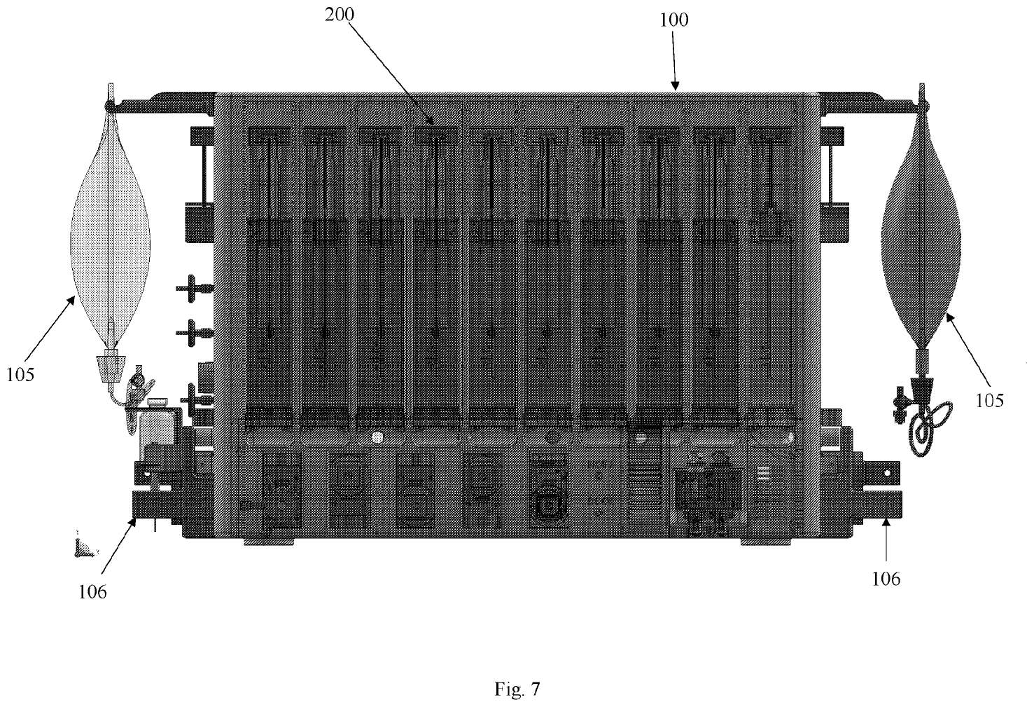

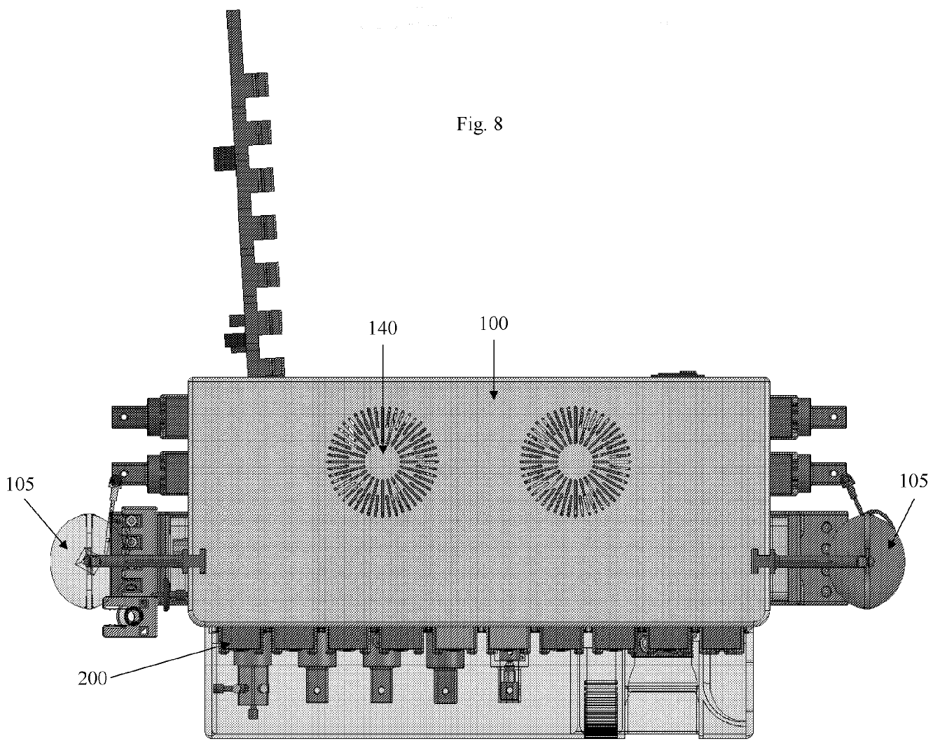

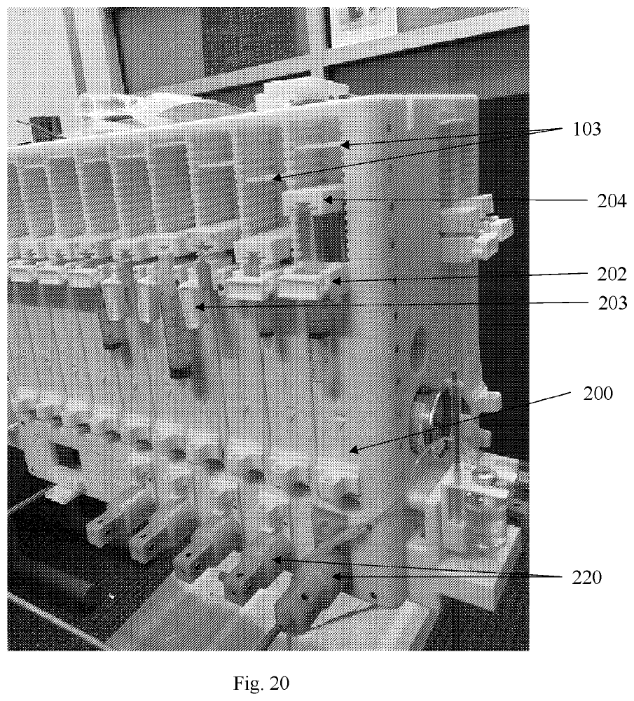

Housing 100 also includes openings for the syringe actuator peripherals (e.g. motor, valves, tubing, etc.). As shown in the exemplary embodiment, these peripheral materials are disposed below the syringe actuators 200. The housing can accommodate a variety of configurations of the actuator peripherals, e.g. the motors and valves (220) can be located below the syringe actuator and positioned in an alternating or staggered configuration in which one motor is higher than an adjacent motor (see FIGS. 4,7 and 37-38). This staggered or offset configuration can be advantageous in that it provides spacing for peripheral components (e.g. tubing) and allows for greater ease of access to (e.g. manually) remove/replace each motor or valve. Additionally or alternatively, in some embodiments adjacent motors/valves can be located in a side-by-side configurations (see FIG. 20). The syringe actuator subassembly 200, as well as the associated peripherals, can be removably coupled to the housing 100 via friction fit, and/or with complimentary male/female interlocking features (e.g. tongue & groove mating). In some embodiments, the modular components are secured within the housing via magnets, which can circumscribe the perimeter of the component and/or housing aperture, or be positioned at only at select locations.

The housing 100 also contains the programmable logic controller, power supply, embedded air pump(s) (140) and reservoir. Accordingly, no external gas, storage or input/supply, are required for operation of the presently disclosed synthesizer system. Instead, the synthesizer system disclosed herein is operated by self-contained pneumatic power (e.g. internal compressor tank) contained within the housing 100. Each actuator 200 can directly connect to the embedded air pump(s) within the housing; alternatively the actuators can be coupled to a manifold that serves as a gateway for directing pressurized air to select actuators. For purpose of illustration and not limitation, an exemplary synthesizer housing is approximately 30 inches (width)×15 inches (depth)×18 inches (height), though size and shape can be adjusted as desired to accommodate any desired application.

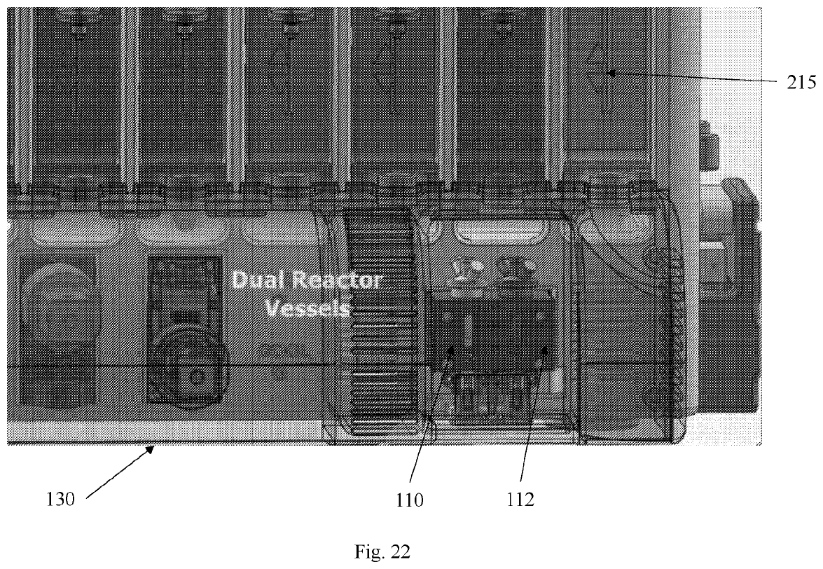

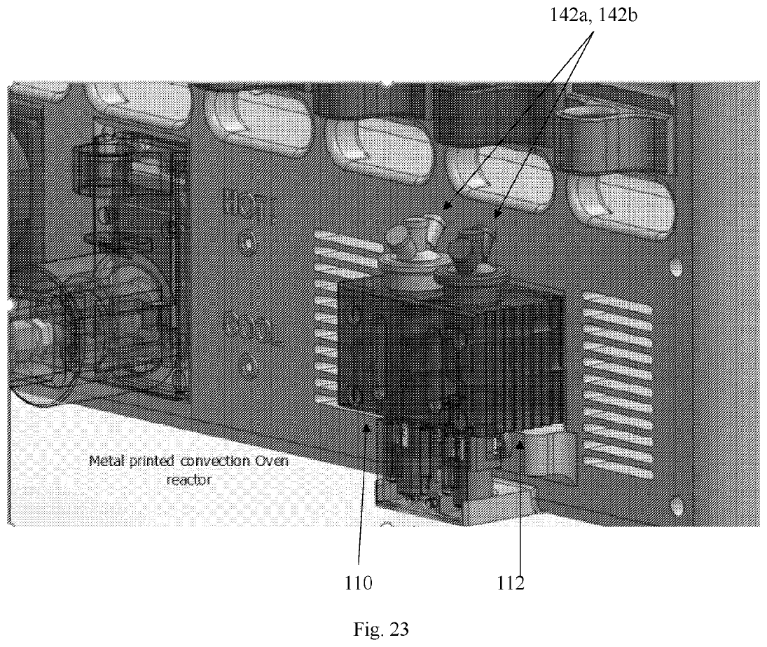

Positioned on one, or both, sides of the housing 101 are bags or pouches containing fluid for flow into and out of the syringe actuators. These bags 105 can be suspended from clips attached to the housing (integrally formed or removable) to maintain a vertical orientation to provide a gravitational supply feed. One, or both, of the bags 105 can contain sterile water for rinsing the system and permitting multiple synthesizing cycles. Additionally, one, or both, sides of the housing 100 can include a receptacle 106 for holding a container (e.g. vial) for delivery of the final solution. Additionally, the present disclosure provides a dual reactor 110, 112 (as shown in FIGS. 22-23) in which each side of the housing 100 can be configured for final product sterility purification allowing for improved efficiency and throughput over existing platforms. These reactors can receive vessels 142a, b which can be independently heated (e.g. approximately 125° C.) and/or cooled (e.g. approximately −10° C.) and can be received within a bracket assembly designed to optimize heat transfer. For example, the bracket containing the dual temperature controlled reactor vessels can include heat sink fins thermally coupled with a thermo Peltier element for rapid cooling. Fan(s) (141) positioned in the top of the housing, and directly above the reactor bracket, can direct airflow against the heat sink to facilitate convective heat transfer. This configuration is advantageous in that it gives the benefit of being able to more quickly process short-lived isotopes. These dual reactors, as well as the remaining peripherals (e.g. motor, valves) can be protected/enclosed with a cover 130. The cover 130 can be formed of a transparent material to allow visual inspection, and a hinge to facilitate easy opening (and removal if desired) to access the underlying components.

Also included within housing 100 are two embedded radiation detectors which can report and quantify the presence of radioactivity. Each side of the synthesizer is monitored by a radiation detector that reports the final dose received in the vial product (disposed at either end 106 of the housing). These radiation detectors can trigger an alarm (visual and/or audible) and record the event when the radiation measurement exceeds a predefined threshold.

In accordance with another aspect of the disclosure, the ergonomic, and modular design allows the user to quickly troubleshoot or replace all parts of the module—without any tooling (i.e. each component can be installed/removed by hand).

Syringe Actuators

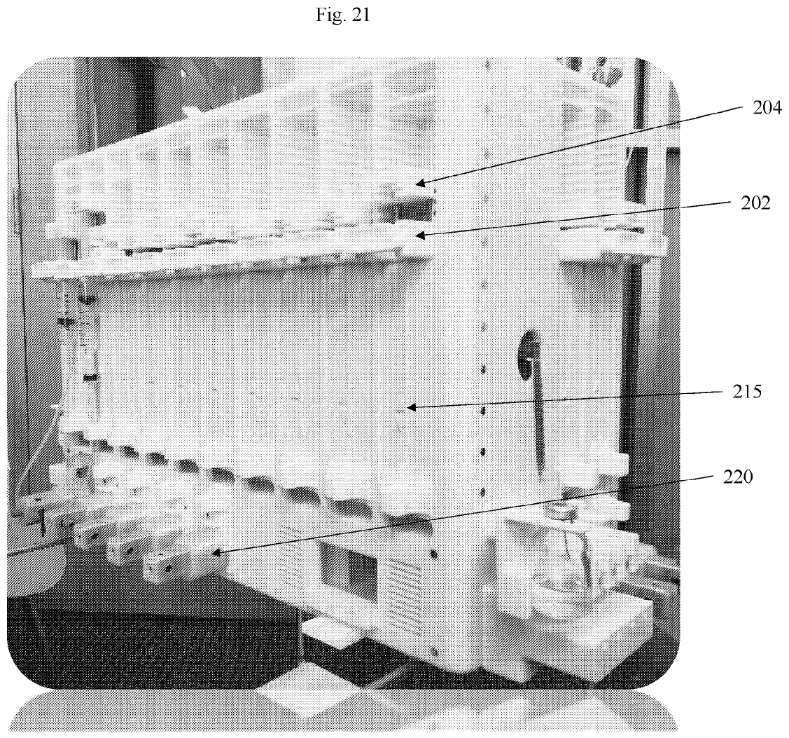

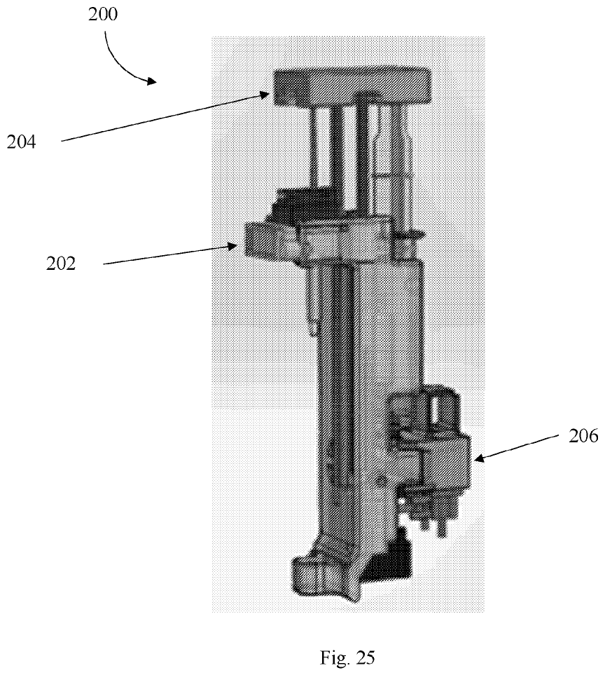

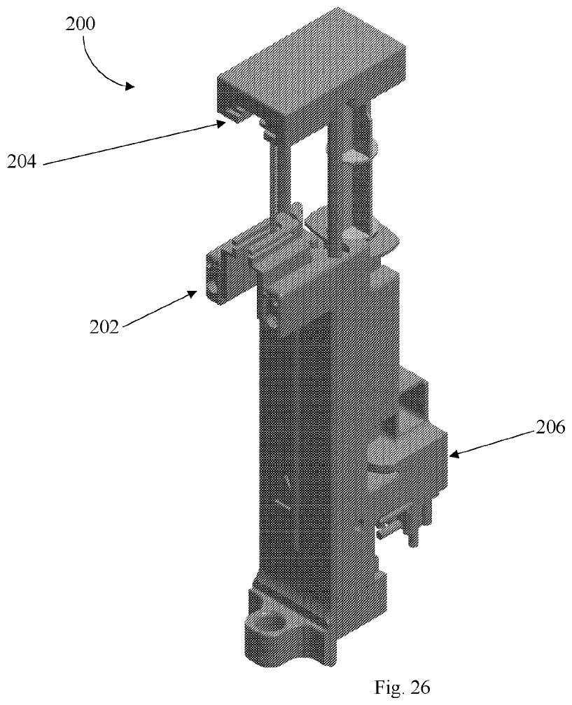

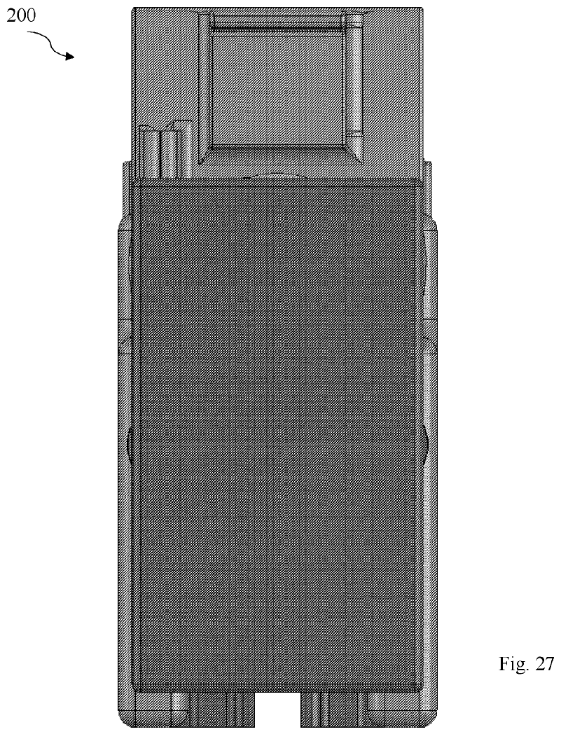

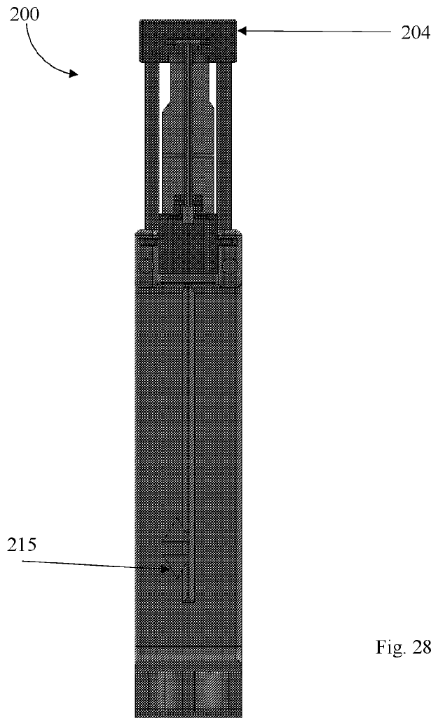

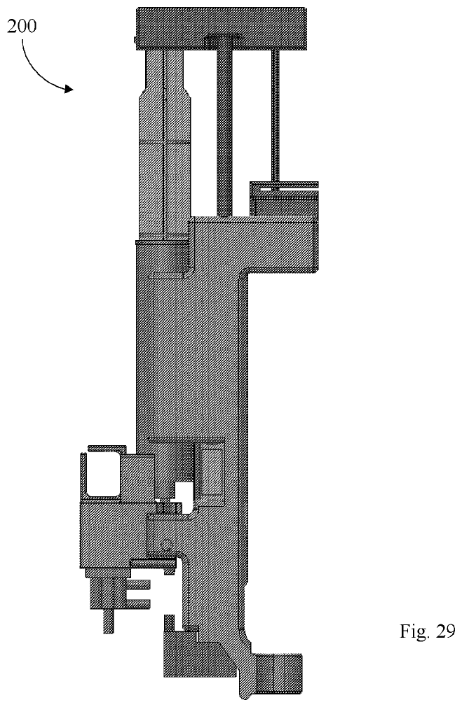

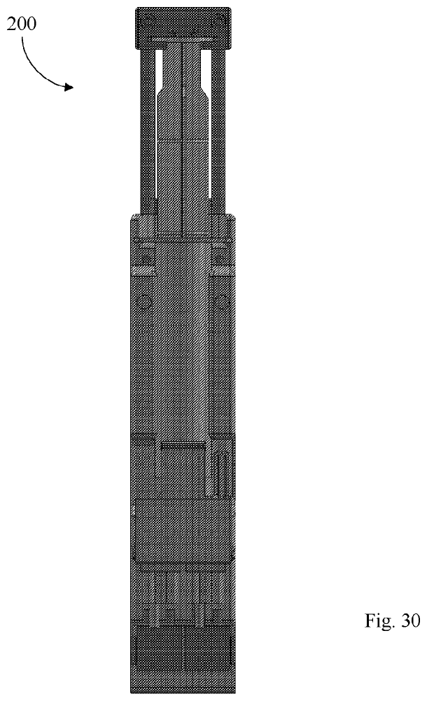

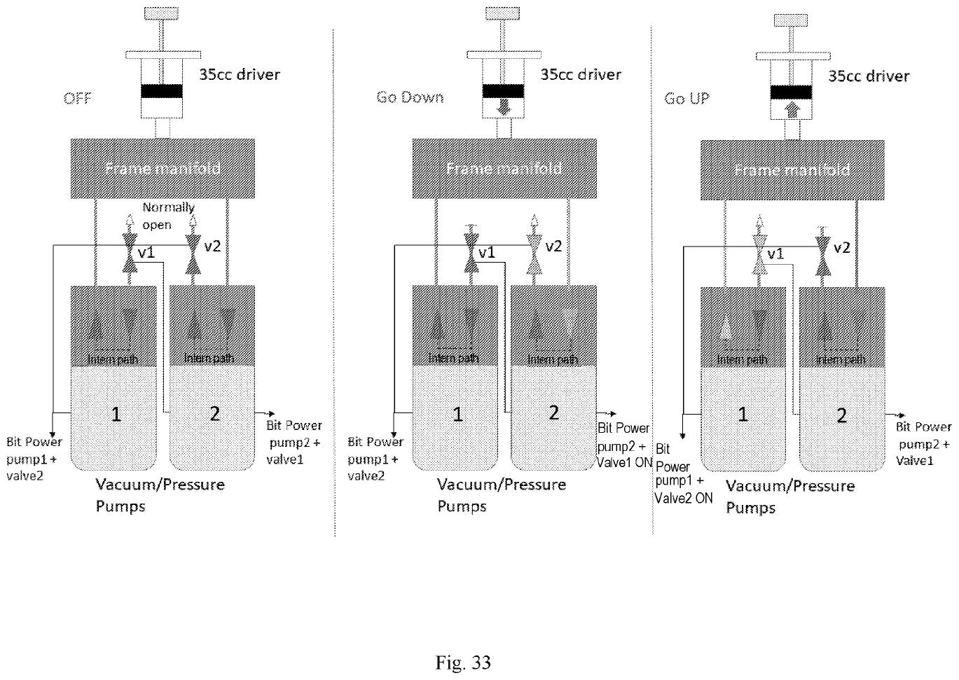

Referring now to FIGS. 20-33 a plurality of modular syringe actuators 200 are provided for installation within the housing 100. Each syringe actuator 200 can include a syringe holder 202 for receiving the syringe. In some embodiments the syringe holder 202 receives the top of the syringe barrel which has flanges which extend radially, or “butterfly” outwards. The syringe holder 202 can be of a fixed geometry (e.g. U-shape) or have biased fingers which grip the syringe barrel for a more secure union.

Also, a door 203 can be included in the syringe holder 202 which can move from an open position to a closed position. For example the door can rotate downward as shown in FIG. 20, to open the holder for receipt of a syringe. Additionally, the external face of the door 203 can include a placard or removable indicia to allow labeling of each syringe actuator so that operators can easily track the progress of a given syringe/solution. In some embodiments, each syringe holder 202 is of a uniform size, with the door 203 serving to securely retain syringes, which may have a smaller diameter than the holder 202 radius of curvature, within the syringe actuator 200. Accordingly, the present synthesizer system can accommodate a plurality of different size syringes for simultaneous use, without the need to change or adjust equipment.

Syringe Actuators 200 also include a driver 204 for engaging and moving the syringe plunger. The driver 204 can include a combination of recess and slot to receive the syringe plunger, with the syringe plunger being inserted from a direction normal to the front face of the driver 204. This recess/slot design allows for a tight engagement of the driver 204 and plunger to minimize relative movement or shifting between the driver 204 and plunger. This maximizes both the efficiency of the system and the range of motion for the plunger. During operation of an upward stroke (to withdraw/load fluid into a syringe barrel), the driver 204 extends upwardly until engaging the stop 103 which precludes further upward movement. In some embodiments, the upward (and/or downward) strokes of the syringe actuators 204 are performed at differing intervals, speeds and/or to differing limits/positions. In some embodiments, all syringe actuators 204 perform uniform upward/downward strokes.

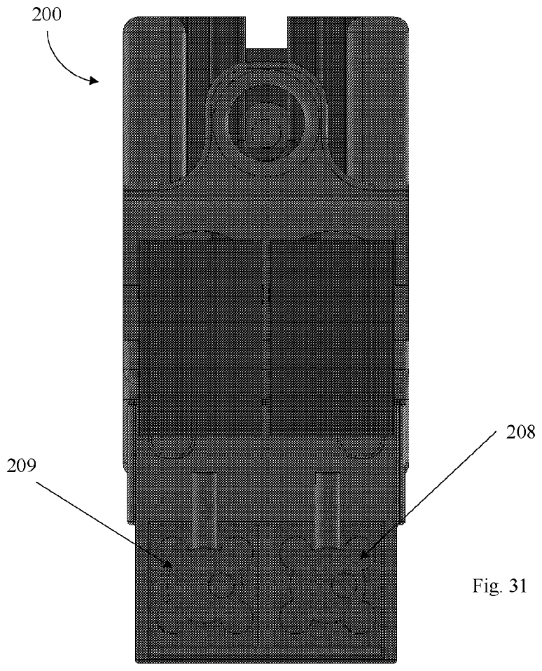

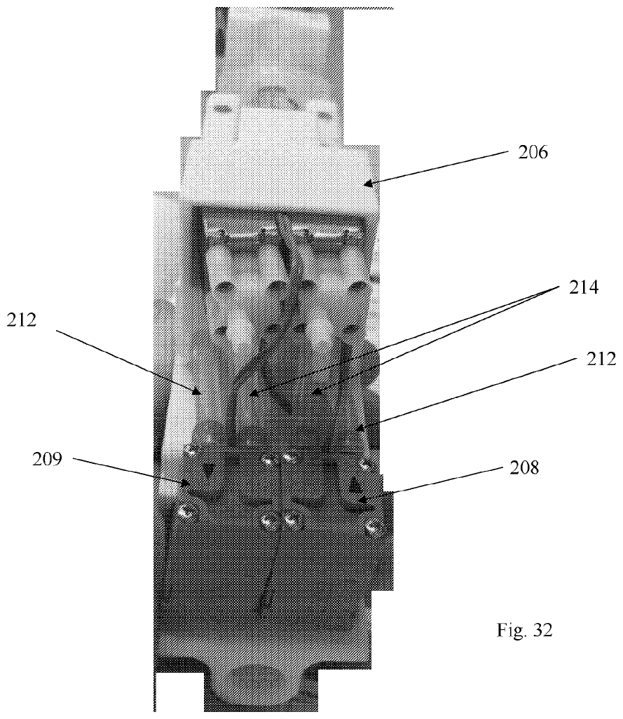

The rear side of syringe actuators 200 includes driver canister/volume 207, and a manifold 206 in direct fluid communication, via conduits 212, with a pump 208 and vacuum 209 source (as shown in FIGS. 31-32). Additionally, the pump 208 and vacuum 209 are in direct fluid contact, via conduits 214, with a switch valve. Accordingly, pump 208 and vacuum 209 are interchangeable/reversible in that either can serve as the pump (i.e. provide a positive pressure differential) or a vacuum (i.e. provide a negative pressure differential), as desired, thereby increasing the design flexibility of the present disclosure. Accordingly, these pumps and vacuum sources generate the fluid flow either into the syringe, or out of the syringe, depending on the particular mode of operation selected. The syringe actuators 200 also include indicators 215 (e.g. LEDs) on the front face which illuminate to show the direction of fluid flow through the system (e.g. when the up arrow is illuminated the syringe plunger is displaced upwards by syringe actuator driver 204 to draw fluid into the syringe barrel; when the down arrow is illuminated the syringe plunger is displaced downwards by syringe actuator driver 204 to dispense fluid out of the syringe barrel). The system can be iterated through as many cycles as desired, with the direction of fluid flow (i.e. withdrawal into the syringe, or dispensing out of the syringe) controlled via the graphic user interface and/or mechanical control. Control of the fluid direction (i.e. loading/dispensing) can be performed by interaction with the graphic user interface. Furthermore, the components (e.g. pump 208, vacuum 209 and switch valve) in manifold 206 do not need to be removed for cleaning. The sterile water contained in bag(s) 105 can be circulated through the synthesizer (tubing and valves) to sanitize the fluid path between operations.

In operation, the driver canister/volume is pressurized to either push the driver 204 upwards thereby drawing fluid into the syringe, or depress the driver 204 downwards to dispense fluid out of the syringe. Also, the plurality of syringe actuators 200 can be operated simultaneously, or independently, as desired. A valve is also provided which can release overhead pressure to stop operation of the driver 204. A potentiometer is also included which can provide continuous, real time feedback of the volume remaining in the syringe and/or driver canister 207. In accordance with an aspect of the present disclosure, the syringe actuator 200 runs at maximum stroke speed for any syringe configuration. In some embodiments, the stroke speed can vary, e.g., the beginning or ending of a stroke can be performed at an alternative (faster or slower) speed than the mid portion of the stroke.

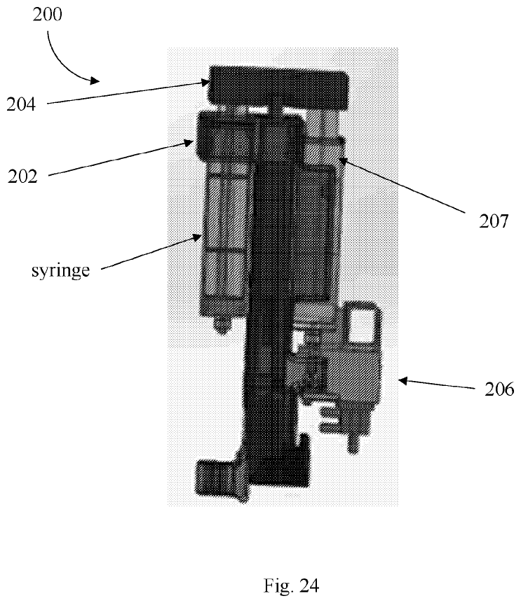

FIG. 24 depicts a logic pathway of the three different stages of operation of the syringe actuator 200 (off shown in the left view; downward stroke or dispensing shown in the middle view; upward stroke or loading shown in the right view). In the off position (left view), the valves (V1, V2) are in the open position, with interchangeable vacuum/pressure pumps 1,2 connected to respective valves. During the downward stroke (middle view), the valve V1 is closed while valve V2 is open and in fluid communication with the vacuum/pressure pump to generate a downward force on the syringe actuator driver 204 and dispense the contents of the syringe. During the upward stroke (right view), the valve V2 is closed while valve V1 is open and in fluid communication with the second vacuum/pressure pump to generate an upward force on the syringe actuator driver 204 and load the contents into the syringe.

In an exemplary embodiment, nine rotary vales are included, each capable of selecting seven distinct positions (each with distinct plumbing/tubing)—for any configuration of syringe actuators employed. FIG. 4 depicts an exemplary valve configuration in which the distal end includes a plurality (e.g. seven) planar facets with exemplary ports 222 extending perpendicularly from the valve 220 (the remaining three ports are not depicted for clarity). These ports 222 can be sized as desired to accommodate the tubing appropriate for the particular radioisotopes being handled by the synthesizer disclosed herein.

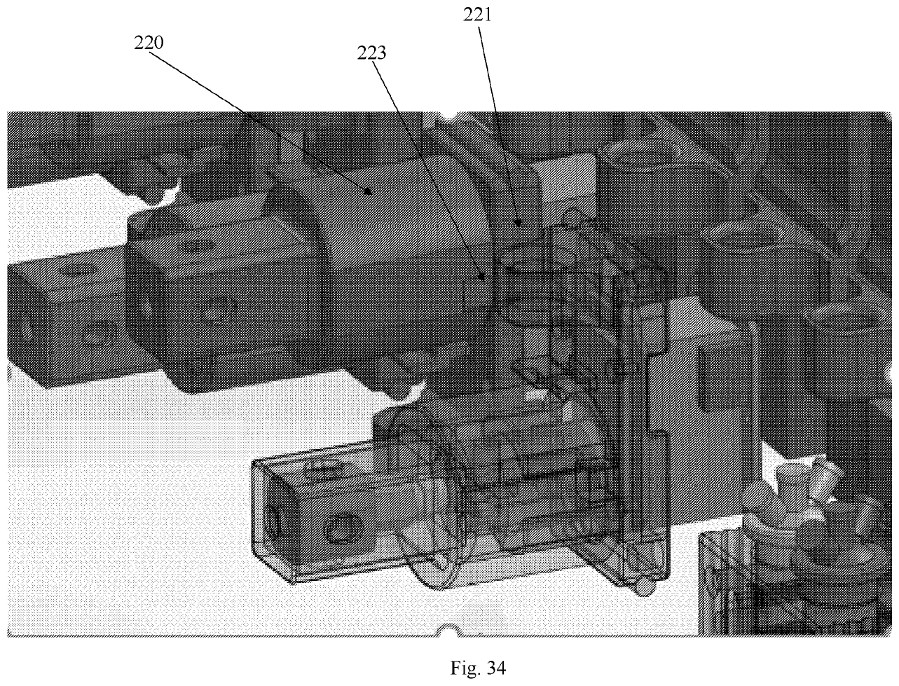

Similarly to the syringe actuators 200, these rotary valves are modular in design (i.e. can be interchangeable in multiple locations in the housing 100) and can be High Performance Liquid Chromatography (HPLC) controlled valves which combine multiple fluidic paths in a single manifold, thereby reducing redundant fluid pathways. The valves, which can be servo valves which adjust fluid flow in proportion to the electrical signal that it receives, and motors are contained within modular casings 220, as shown in FIGS. 4 and 20. The casings 220 (which can also be fabricated from 3-D printing of nylon) can include a locking feature for coupling to the panel 221 (which can be formed of metal, e.g., aluminum). As shown in FIG. 34, the locking feature can include biased tongs/fingers 223 which are received within complimentary shaped recesses of casing 220.

Significantly, the present synthesis system does not require solenoid valves along the fluid path, nor stepper motors for operation. Accordingly, the present system is lighter, draws less power, and provides a more reliable operation than conventional synthesizers which rely on such solenoid valves to control fluid flow.

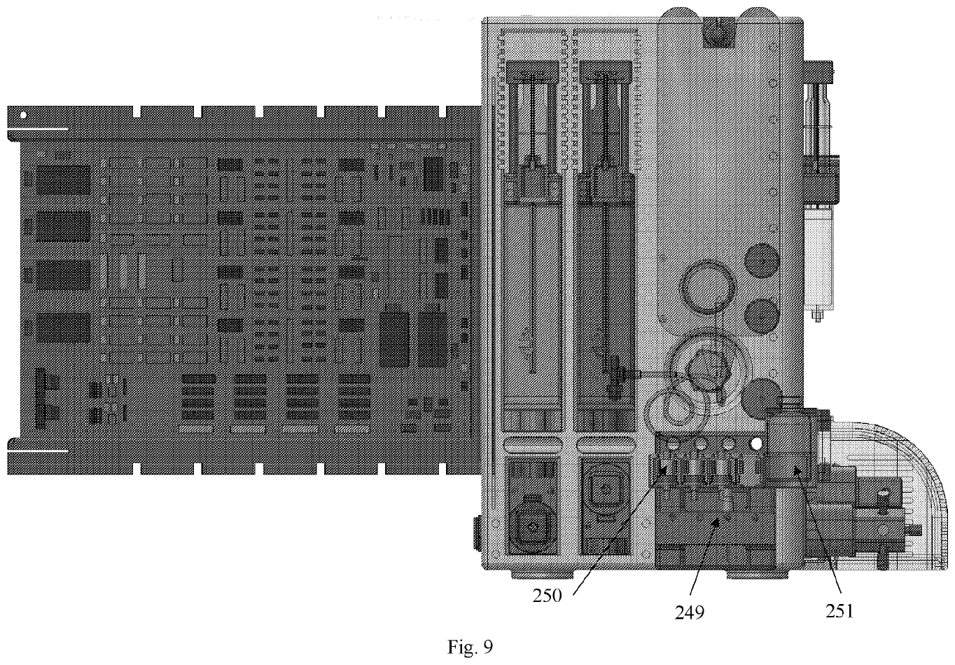

On either, or both, sides of the housing a shelf or bracket is provided for holding the target material 250 generated from the cyclotron operation. In the exemplary embodiment shown in FIG. 9, the bracket 249 holds three vials which can contain distinct target material, as well as a vial 251 for delivery of the final solution—post synthesis (note: the tubing fluidly coupling the vials to the actuators, valves, etc. are omitted for clarity).

Graphical User Interface

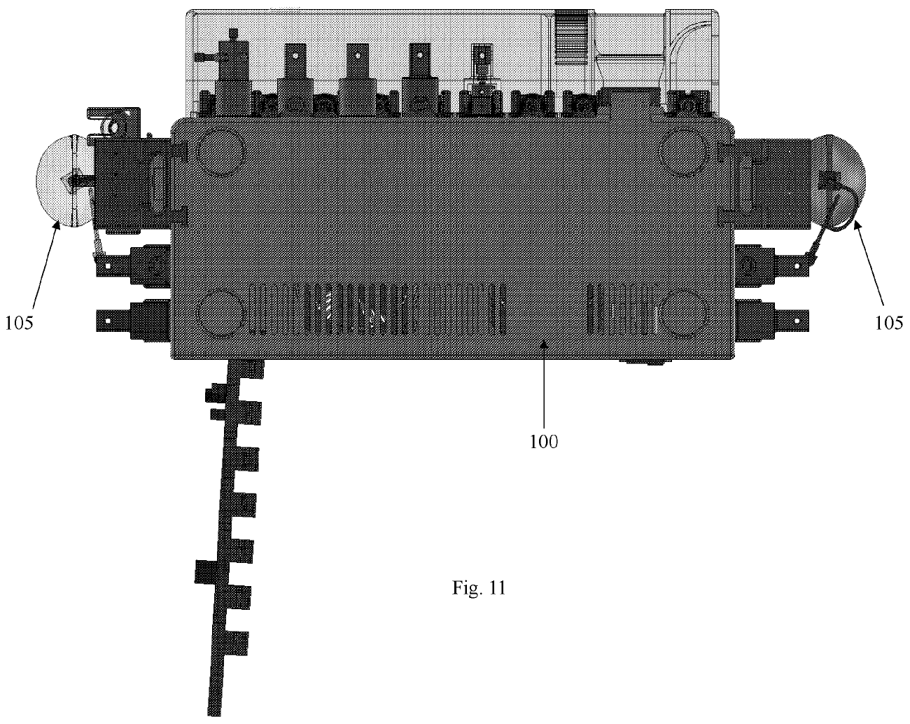

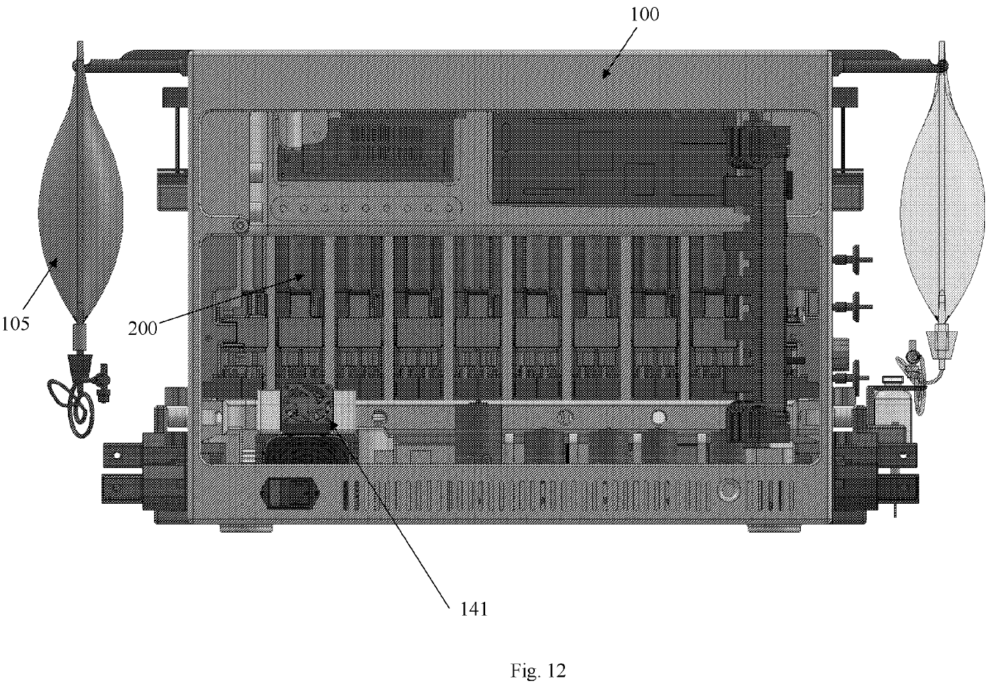

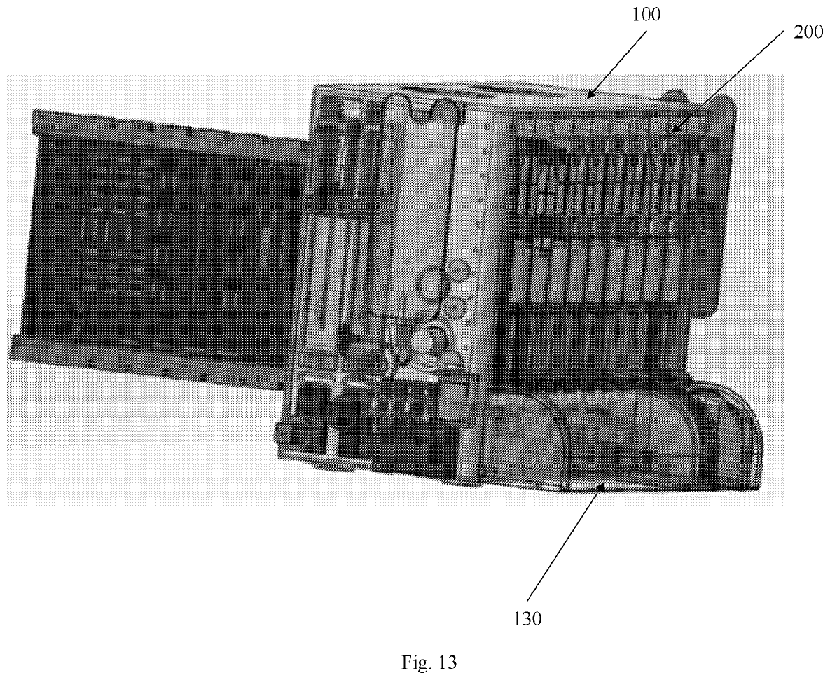

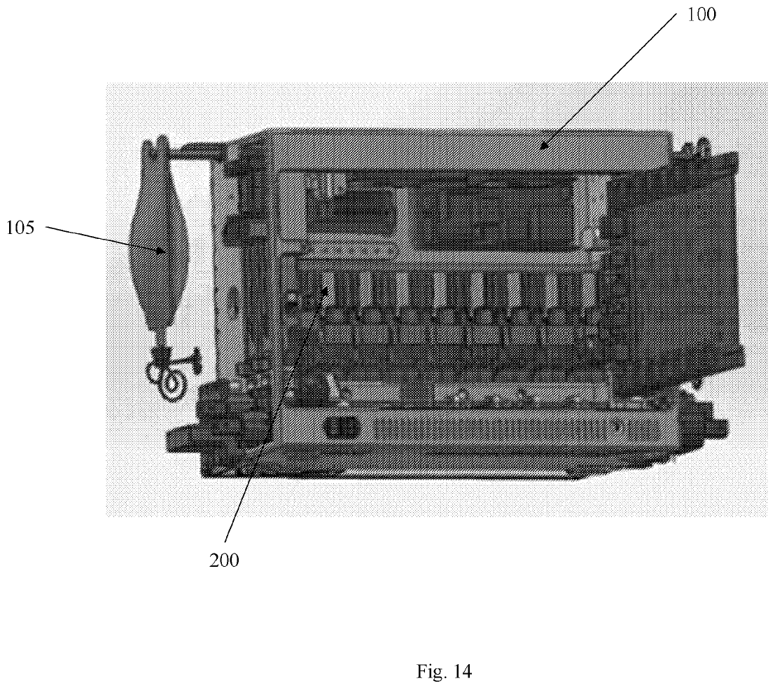

In accordance with an aspect of the present disclosure, an embedded custom microprocessor printed circuit board (as shown in FIGS. 9-13) fitted within housing 100, provides automated synthesis module for development and manufacturing of novel radiometallic tracers. The programmable microprocessor can run multi compound methods (no code program needed) and runs a logical script list created within the application software. Additionally, a memory is provided for saving methods and run reports in compliance with C.F.R. Title 21 part 11 guidelines for data security.

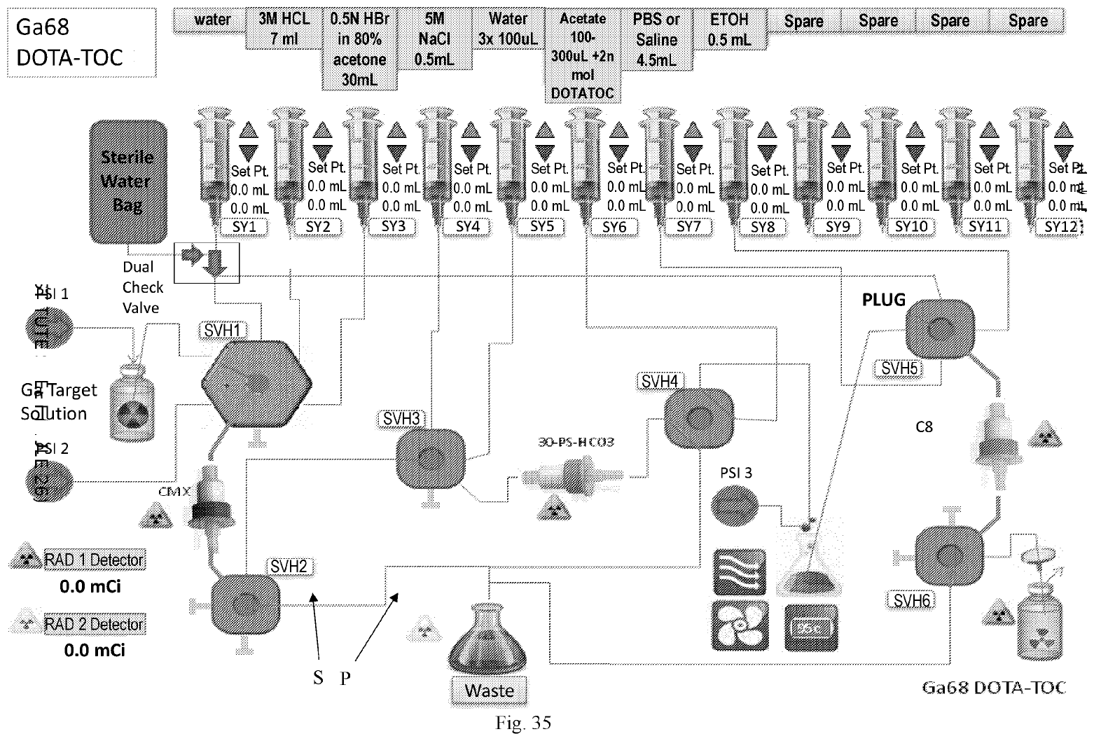

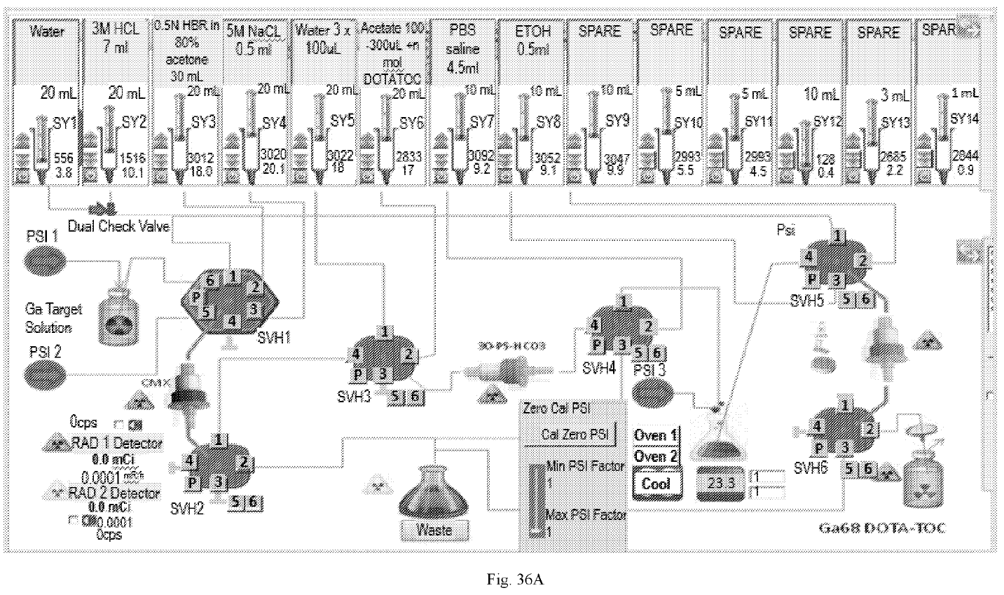

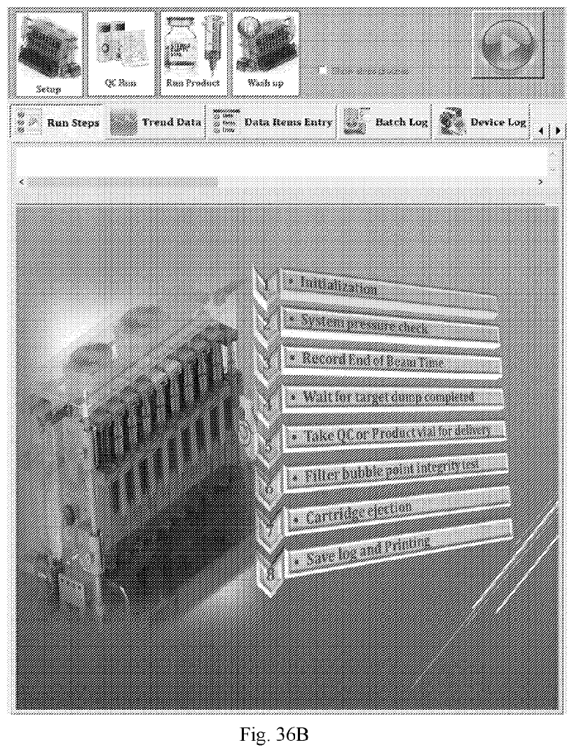

A graphical user interface (GUI) is provided which allows for dynamic interaction between user and hardware units. As shown in FIGS. 35-36B, the GUI presents the user with four steps, Setup, QC Run, Run Product and Washup, as shown in the top right of the exemplary screenshot shown in FIG. 36A-B. This system provides for a one-time setup for complete production including batch record log; Quality Control samples are drawn remotely; and the program performs a filter integrity test at the end of each run (before generating a full production batch report). Filter integrity testing of the final sterile product is also part of the automated process, thus reducing personnel exposure due to radiation handling.

As shown in FIGS. 35-36B, a status indicator is presented for each of the fourteen modular syringe actuators depicting, e.g., contents of the syringe, remaining volume, and fluid path including position of rotary valves. During operation, the fluid flow is routed through pathway(s) determined by the programmable circuit. As shown in the exemplary embodiment of FIG. 35, twelve syringes (labeled “SY1”-“SY12”) are loaded within the syringe actuators 200, with the syringes having differing contents and volumes contained therein, and some syringes being empty (labeled “spare”) at the outset (as shown in the rectangular labels, e.g. “water”, “3M HCL 7 ml”, etc.) at the top of the figure). The plumbing lines “P” and valve positions/switches “S” indicate the particular fluid flow for this exemplary embodiment. The valves can switch (e.g. rotate the conduit “S”) from, e.g. fluidly coupling with the Ga target solution container/vial, and the syringes “SV1”, etc. as shown. A plurality of pumps “PSI 1”-“PSI 3” are provided to drive the fluid flow and a plurality of Radiation detectors “RAD 1” and “RAD 2” are distributed throughout the fluid flow to monitor radiation levels and signal any irregularities or readings beyond acceptable thresholds.

Additionally, FIGS. 36A-B depict exemplary views of isolated window panes presented in a GUI during operation of the system. FIG. 36A depicts the plumbing configuration for the particular embodiment, illuminating and enumerating the seven different positions for the switch valve “SVH1” to fluidly connect with the various syringes and/or target solution and pressure source “PSI 1”. FIG. 3B depicts a workflow and picture of the synthesizer system.

The modular synthesizer and automated process disclosed herein can be employed to produce unlimited types of radio metal tracers. For purpose of illustration and not limitation, exemplary radioisotopes such as ⁶⁸Ga, ⁶⁴Cu and ⁸⁹Zr can be radiolabeled using the system and techniques disclosed herein.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A modular radio-labeling tracer synthesizer system comprising:
a housing, the housing having at least one slot;
at least one syringe actuator, the at least one syringe actuator disposed within the slot and removably attached to the housing; and
at least one servo motor and at least one rotary valve, the at least one servo motor and at least one rotary valve removably attached to the housing.

2. The system of claim 1, wherein the at least one syringe actuator is attached via magnet(s).

3. The system of claim 1, wherein the at least one rotary valve is removably attached to the at least one servo motor.

4. The system of claim 1, wherein the at least one syringe actuator includes a syringe driver configured to engage a syringe plunger for displacement in a loading and dispensing direction.

5. The system of claim 1, wherein the at least one syringe actuator includes a syringe holder, the syringe holder configured to receive a variety of syringe sizes.

6. The system of claim 5, wherein the syringe holder includes a door which can move from an open position to a closed position.

7. The system of claim 4, wherein the housing includes a stopper, the stopper limiting displacement of the syringe driver.

8. The system of claim 1, wherein the housing includes fourteen slots, with a syringe actuator disposed in each slot.

9. The system of claim 1, each rotary valve(s) has seven positions.

10. The system of claim 1, wherein the housing includes nine rotary valves.

11. The system of claim 1, wherein the housing further comprises dual temperature controlled reactor vessels.

12. The system of claim 1, wherein the housing further comprises a cooling element.

13. The system of claim 1, wherein the housing further comprises a compressor.

14. The system of claim 1, wherein the housing further comprises at least one radiation detector.

15. The system of claim 1, wherein the housing further comprises at least one programmable microprocessor.

16. A syringe actuator, the syringe actuator comprising:
a syringe holder,
a syringe driver,
a manifold, the manifold having:
a pump source,
a vacuum source,
a first conduit in fluid communication with the pump source, second conduit in fluid communication with a vacuum source, and
a switch valve, the switch valve configured to direct fluid flow through at least one of the conduits.

17. The syringe actuator of claim 16, further comprising a third conduit connecting the switch valve and pump source in fluid communication.

18. The syringe actuator of claim 16, further comprising a forth conduit connecting the switch valve and vacuum source in fluid communication.

19. The syringe actuator of claim 16, wherein the syringe actuator includes a syringe driver configured to engage a syringe plunger for displacement in a loading and dispensing direction.

20. The syringe actuator of claim 18, wherein the syringe actuator includes a visual indicator depicting the direction of displacement of the syringe plunger.
